(19) United States
(12) Patent Application Publication
(10) Pub. No.: US 2017/0197033 A1
(43) Pub. Date: Jul. 13, 2017

(54) ADAPTER, PROCESS AND USE FOR INSTILLATION OF AGENTS INTO THE BLADDER THROUGH THE URETHRA WITHOUT CATHETER

(71) Applicants: Sándor LOVÁSZ, Budapest (HU); Gábor RÉNYI, Budapest (HU); János GIBER, Budapest (HU)

(72) Inventors: Sándor LOVÁSZ, Budapest (HU); Gábor RÉNYI, Budapest (HU); János GIBER, Budapest (HU)

(21) Appl. No.: 15/268,171

(22) Filed: Sep. 16, 2016

Related U.S. Application Data
(63) Continuation of application No. PCT/HU2016/000063, filed on Sep. 15, 2016.

(30) Foreign Application Priority Data
Sep. 16, 2015 (HU) P-1500419
Dec. 23, 2015 (HU) P-1500648

(51) Int. Cl. A61M 5/30; A61M 5/00; A61M 31/00

(52) U.S. Cl. CPC A61M 5/30; A61M 31/005; A61M 5/007; A61M 2005/3103

(57) ABSTRACT

An adapter is provided with a central, longitudinal, inner through hole and has a rounded off tip-part to be inserted into the external urethral orifice, a sealing collar and a cylindrical connecting part known connectable to the appropriate part of a dosing device, advantageously to the ISO 594 standard Luer Slip or Luer-Lock tip of a syringe. The adapter is connected by known method to the appropriate part of a dosing device containing the active and/or contrast agent, then the active and/or contrast agent is ingested leakage-free into the urethra or into the urethra and the bladder through the adapter, then through the external urethral orifice by using overpressure in the dosing device and inserting the tip-part into the external urethral orifice and squeezing the sealing collar in a proper way to the external urethral orifice.

### CROSS-REFERENCE TO RELATED APPLICATION

This application claims the benefit of Hungarian Patent Application No. HU P-1500419, filed Sep. 16, 2015, and of Hungarian Patent Application No. HU P-1500648, filed Dec. 23, 2015, and is also a continuation of PCT International Patent Application No. PCT/HU2016/000063, filed Sep. 15, 2016, each of which is hereby incorporated by reference in its entirety.

### BACKGROUND OF THE INVENTION

The subject matter of our invention is an adapter, advantageously single use, advantageously cylindrical, advantageously syringe adapter, made out of solid, pharmaceutically applicable, advantageously for human therapy applicable, advantageously rigid or flexible material, for injection of active and/or contrast agent in liquid, fluid-like, gas or colloid disperse form, painlessly or by reduced pain, without leakage into the urethra or into the urethra and the bladder through the external urethral orifice without using catheter.

The generally accepted and widespread practice in cases of various diseases of the bladder is direct injection of the solution with curative effect, or solution of drug or mixture of these solutions (so called “bladder cocktails”) directly into the bladder generally through a catheter introduced into the urethra. One of the targets of the treatment is to reproduce and heal the so called “GAG” (glycosaminoglycan) layer, which is a defending mucous-layer on the surface of the inner wall of the bladder.

Thus the active ingredients arrive into the bladder in higher concentration than being orally or parenterally administered bypassing the intestinal tract by injection. As only a fraction of the administered dose of drug is absorbed and gets into the circulation in this way the risk of emerging undesirable or even dangerous side effects can also be minimized.

For the local treatment of the bladder (bladder-instillation) with curative active ingredients or drugs, all accessible special medical literature, professional guidelines as well as textbooks recommend the method with catheter as the only possible method.

This generally accepted method however has several significant disadvantages:

Prior to applying the method with catheter a lubricant gel has to be applied rendering the method more expensive and the process more complicated. In more than half of the cases of interstitial cystitis (bladder pain syndrome) the urethra itself is also involved in the disease and therefore it is hypersensitive for contacting or pressing it and often it can even cause expressed pain, making patients' sexual life intolerable as well as sitting and walking. In such cases applying the catheter in the urethra may cause severe pain resulting in refusal of treatment.

The straight and relatively inflexible draining catheters straighten the bends of the urethra thereby scraping along one or the other side of the surface of the inner wall of the urethra, causing minimally microscopic superficial mucous membrane lesions but sometimes also major lesions with macroscopic bleeding (urethrorrhagia) can be seen.

Due to the anatomic differences between the genders, the longer urethra in men with several natural bends is even more frail and during the course of the treatment (lasting 10 to 15 weeks with regular, weekly one catheterization) the repeated iatrogenic lesions at the same points of the urethra may even result urethral stricture caused by scar tissue as tardive complication.

The drugs administered by catheter remain until the following urination in the bladder because in the moment of pulling out the catheter, the natural muscular closing system of the urethra (internal and external sphincter) closes and therefore no active ingredient gets into the urethra.

Therefore the task of the invention is to develop a method by which the solution comprising the active ingredient can be injected into the bladder without catheterization of the urethra and at the same time a smaller quantity of drug, but with effective concentration can remain in the urethra. Direct injection into the urethra seemed to be a good solution. However regarding the difference between the internal diameter of the external urethral orifice and the outside diameter of the Luer-lock nozzle, tip of the syringe used for injection, a significant loss of liquid can occur by leakage hindering patients of both genders being such interventions successful.

The adequate application of the adapter according to the invention provides a solution of these troubles and difficulties.

### SUMMARY OF THE INVENTION

Disclosed is an adapter for ingestion advantageously injection, instillation of active and/or contrast agent advantageously drug solution and/or diagnostic solution painlessly or by reduced pain, without leakage into the urethra or into the urethra and the bladder through the external urethral orifice without using catheter.

The 6 adapter is provided with a 5 central, longitudinal, inner through hole and has a rounded off 1 tip-part to be inserted into the external urethral orifice, a 2 sealing collar and a 3 cylindrical connecting part known connectable to the 4 appropriate part of a dosing device, advantageously to the ISO 594 standard Luer Slip or Luer-Lock tip of a syringe.

The form of the end of the 1 tip-part of the adapter according to the invention inserted into the external urethral orifice is specially rounded off, softened, advantageously cylindrical, further advantageously conical, and is characteristic for the invention and has unique parameters.

Furthermore the adapter according to the invention fits generally and perfectly to the external urethral orifice.

The material of the adapter according to the invention is solid advantageously rigid or flexible, pharmaceutically applicable advantageously for human therapy applicable material, especially advantageously metal which may be especially advantageously copper, bronze, aluminum and stainless steel or any alloy of them, further advantageously glass, further advantageously plastic, which may be especially advantageously thermosetting, thermoplastic or flexible plastic, thermoset elastomer, especially advantageously silicone, thermoplastic elastomer, especially advantageously thermoplastic polyurethane, further advantageously synthetic rubber or synthetic rubber like material, or synthetic rubber or synthetic rubber like based material, especially advantageously polytetrafluoroethylene (Teflon, PTFE), polyethylene, polypropylene, polystyrene, polyester, polylactic acid, polycarbonate, polyvinyl chloride, polyethersulfone, polyacrylate, hydrogel (acrylate), polysulfone, polyetheretherketone (PEEK), poly-p-xylylene (parylene), fluoropolymer, further advantageously any natural based rigid or flexible material, especially advantageously latex or latex based, or natural based rubber or rubber like material or rubber or rubber like material based material, further especially advantageously plastic or alloy or mixture of any of the above listed materials, further advantageously any other pharmaceutically applicable advantageously for human therapy applicable, for mass producing, for quantity production in series suitable material and by technology for mass producing, for quantity production especially advantageously by die casting or injection molding technology, or 3D printing in series producible material, which can be used for producing the adapter according to the invention.

Using the adapter according to the invention, the subject matter of our invention is furthermore a non-invasive, humane, catheter-free, leakage-free process for injection of active and/or contrast agent in liquid, fluid-like, gas or colloid disperse form, advantageously in aerosol, emulsion, gel, foam or suspension form into the urethra or into the urethra and the bladder through the external urethral orifice from dosing device, advantageously syringe, by using overpressure, advantageously by using manual or gravity pressure, without using catheter.

By the process according to the invention the 6 adapter is connected by known method to the 4 appropriate part of a dosing device containing the active and/or contrast agent, advantageously to the ISO 594 standard Luer Slip or Luer-Lock tip of a syringe, then the active and/or contrast agent is injected leakage-free into the urethra or into the urethra and the bladder through the adapter, then through the external urethral orifice by using overpressure in the dosing device and inserting the 1 tip-part into the external urethral orifice and squeezing the 2 sealing collar in a proper way to the external urethral orifice.

The process according to the invention is for both genders adults and children, advantageously for women, men and children applicable.

The dosing device used in the process according to the invention can be used for injection active and/or contrast agents for local treatment or for diagnosis of bladder and/or urethra by using overpressure into the urethra and the bladder through the external urethral orifice.

Using the adapter and process according to the invention, catheter-free, painless or pain-reduced and leakage-free advantageously bladder filling, bladder rinsing, bladder diagnosis, especially advantageously intravesical (bladder) instillation can be implemented.

According to the invention by the injection of the active and/or contrast agents into the bladder through the external urethral orifice and urethra by using the adapter and catheter-free process according to the invention, direct local treatment and diagnosis of the urethra can be implemented, which is impossible by using catheter.

Furthermore the subject matter of the invention is the use of the adapter and process according to the invention for injection of active and/or contrast agent in liquid, fluid-like, gas or colloid disperse form, painlessly or by reduced pain, without leakage into the urethra or into the urethra and the bladder through the external urethral orifice without using catheter.

During the use according to the invention, the injected active agent is advantageously solution of curative active ingredient or of drug, or a mixture of them, the so called “bladder cocktail” for local, advantageously local curative or pharmaceutical treatment of bladder and/or urethra.

The adapter and catheter-free process according to the invention can be used advantageously for painless or pain-reduced bladder filling, bladder rinsing, especially advantageously for intravesical (bladder) instillation, namely for the local pharmaceutical painless or pain-reduced treatment of the bladder and further especially advantageously for the local painless or pain-reduced pharmaceutical treatment of the urethra.

The adapter and process according to the invention can be used further advantageously for injection of agent in liquid, fluid-like, gas or colloid disperse form especially advantageously in liquid form, comprising active and/or contrast agent, advantageously solution of curative active ingredient or drug, further advantageously diagnostic solution comprising contrast agent, through the external urethral orifice and urethra into the bladder, advantageously for local treatment of bladder and/or urethra especially advantageously local treatment of interstitial cystitis, bladder pain syndrome, further especially advantageously with analgesic effect, further especially advantageously for local anesthesia, further especially advantageously for local treatment of low urinary tract symptoms, further especially advantageously for local analgesic after-treatment of patients irradiated for prostate cancer, bladder cancer or pelvis tumor, further especially advantageously for local chemo-therapeutical treatment of female patients suffering in bladder cancer

Using the adapter and process according to the invention the agent can be injected safe, catheter- and leakage-free for the above indications, advantageously for local treatments and diagnostic targets.

### BRIEF DESCRIPTION OF THE DRAWINGS

FIG. 1A shows a top view of the adapter of the invention.

FIG. 1B shows one side view of the adapter of the invention.

FIG. 1C shows another side view of the adapter of the invention.

FIG. 1D shows a bottom view of the adapter of the invention.

FIG. 2 shows use of the adapter in a Luer Slip fitting.

FIG. 3 shows use of the adapter in a Luer Lock fitting.

### DETAILED DESCRIPTION OF THE INVENTION

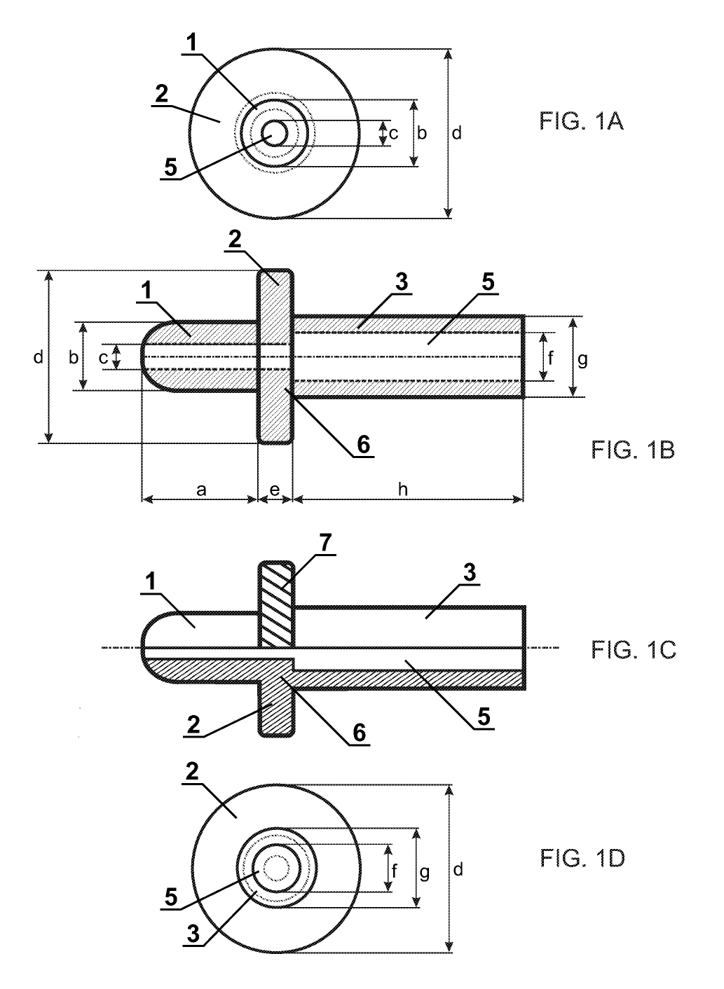

According to the definitions and notations of the FIGS. 1 to 3, the adapter according to the invention can be described as follows.

In FIG. 1 enclosed to the specification, the 6 adapter according to the invention is presented in three views, namely top-view, side-view and bottom view.

The side view is presented in two versions: in a basic segment version and in a half view—half segment version.

The 6 adapter according to the invention has three parts as clearly shown in the side view of FIG. 1: the 1 tip-part fitting directly to the urethra through the external urethral orifice, the 2 sealing collar preventing leakage of agent, advantageously liquid injected into the urethra, and the 3 cylindrical connecting part, connecting to the 4 appropriate part of the dosing device, advantageously to one of the type of ISO 594 standard Luer Taper tip, either to Luer Slip or to Luer-Lock tip of the syringe by known method, advantageously precisely connected or built on.

Concerning Luer Slip fitting, the 3 cylindrical connecting part of the adapter is connected by a standard conic tip, concerning Luer Lock fitting, the connection is additionally also fixed by thread or a bayonet lock in case of high pressure injection.

As in our case low pressure is used, according to the solution of our invention, such a fixation is not needed even concerning Luer Lock fitting, so the 3 cylindrical connecting part can be simply fitted to the Luer Lock tip also by pressing together and held by friction similar to Luer Slip fitting.

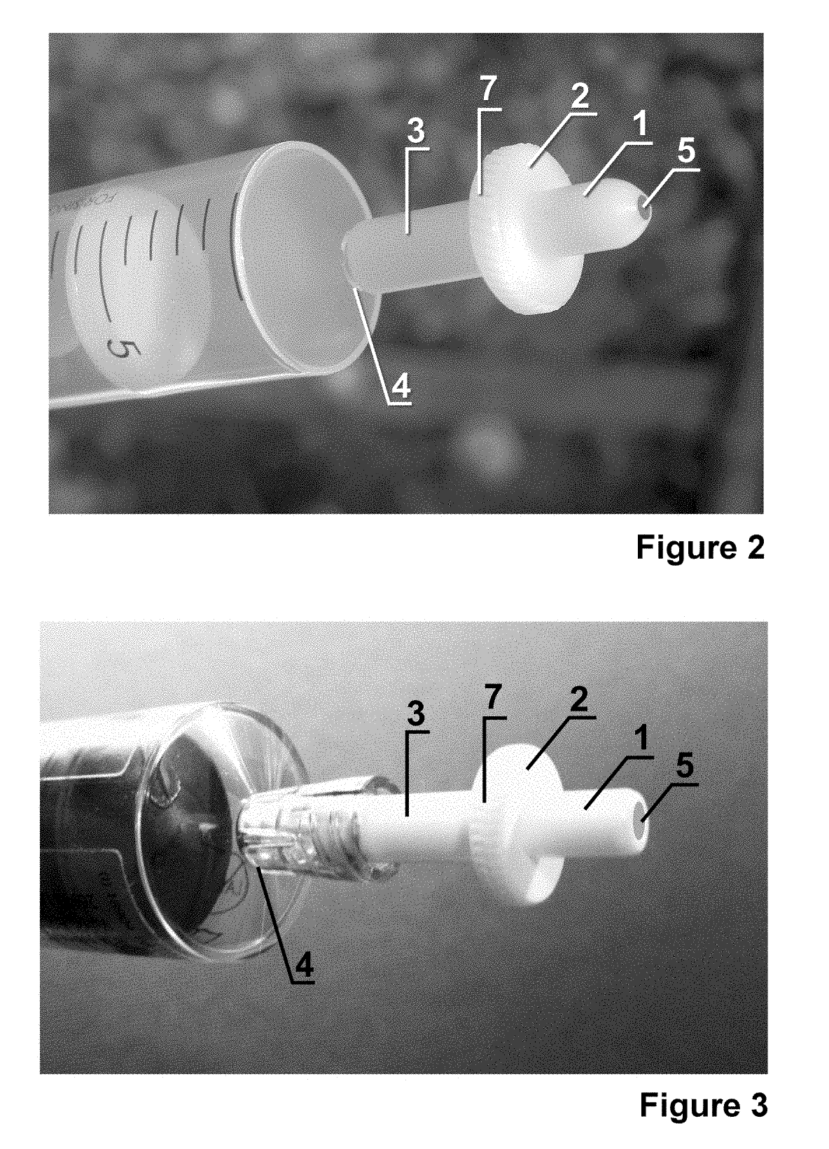

In FIG. 2 Luer Slip fitting and in FIG. 3 Luer Lock fitting are presented, and is also shown and signed in both Figures, that according to the solution of the invention the external circumference of the 2 sealing collar is 7 knurled to facilitate adjusting and removal to the external urethral orifice by hindering the slipping.

As clearly shown in all three views of FIG. 1, the 6 adapter according to the invention is provided with a 5 central, longitudinal inner through hole through which the agent, advantageously the liquid gets from the dosing device, advantageously from liquid dosing device, advantageously from syringe, further advantageously from infusion device through the 1 tip part and 3 cylindrical connecting part into the external urethral orifice and then into the urethra. The parts of 5 central, inner through hole of 1 tip-part and of 3 cylindrical connecting part of the 6 adapter according to the invention have advantageously different “c” and “f” diameters.

An important characteristic of the adapter according to the invention and also presented in top and side view of FIG. 1 is, that the 1 tip-part to be inserted into the orifice of the urethra is rounded off specially, its surface, and edges are softened, thus the inner wall of the urethra would be not injured even if a precise coaxial insertion cannot be achieved.

The characteristic sizes of the different parts of the adapter according to the invention are signed by small letters in the figures.

According to the side view of FIG. 1 the “a” length of the 1 tip-part to be inserted into external urethral orifice is advantageously 8 to 12 mm especially advantageously 10 mm to assure safe fitting and its “b” diameter is advantageously 6 mm (18 French) corresponding to an average catheter diameter and can be inserted in the overwhelming majority of cases without difficulty into the urethra.

As can be seen in the top view of FIG. 1 the advantageous “c” diameter of the 5 central, longitudinal inner through hole of the 1 tip-part to be inserted into the urethra is advantageously 1.8 mm to 3.5 mm, especially advantageously 2.5 mm so that a thick gel-like solution or suspension can be injected with small resistance into the urethra.

A further characteristic of the invention is, that the edges of the tip of the hole are softened by fine machining.

The end of the 1 tip-part according to the solution of the invention has a softened shape, so, that no sharp edges causing mucosal lesion should remain. The round-off radius at the 1 tip part's end is no single data, its tolerance is 1 mm downwards (smaller) or 2 mm upwards (bigger). The round off and softening are also applied outward and inward to the 5 central, inner through hole.

According to the top view and bottom view of FIG. 1, the 2 sealing collar of the adapter according to the invention is a disk. The “d” diameter of the disk is advantageously 12 mm to 20 mm, specially advantageously 15 mm and the “e” thickness of the disk is advantageously 2 mm to 5 mm especially advantageously 3 mm as illustrated in the side view of FIG. 1. The disk fits over the roughly round or oval shaped external urethral orifice, and assures perfect sealing without leakage.

The mucous surfaces close to the external urethral orifice are sensitive even to microscopic lesion, therefore the edges of the disk as a solution by the invention are chamfered to 45° or rounded off in a circle advantageously with 1 mm radius and the remained edges of the disk should also be softened.

According to the solution of the invention, as shown in the half-segment-half view version of side view in FIG. 1 and in FIGS. 2 and 3, presenting the Luer Tapers, the external circumference of the 2 sealing collar is 7 knurled to facilitate adjusting and removal to the external urethral orifice by hindering the slipping.

According to the bottom view of FIG. 1 and side-view of FIG. 1, the 5 central, longitudinal internal hole of 6 adapter is shown as hole of 3 cylindrical connecting part. The “f” inner caliber of the 5 central, inner through hole of 3 cylindrical connecting part is advantageously 3.8 mm to 5.2 mm especially advantageously 4.2 mm and “g” outer diameter is advantageously at least 7 mm, specially advantageously 7 mm which characteristic parameters assure the thickness of wall of cylinder for suitable stability.

According to the side view of FIG. 1 the “h” length of the 3 cylindrical connecting part is advantageously at least 20 mm, especially advantageously 20 mm, to assure the required visual control for the person performing the treatment so even dosing devices advantageously syringes with larger diameter (with e.g. 50 ml capacity) cannot hide the adapter being inserted into the external urethral orifice.

The 3 cylindrical connecting part of the 6 adapter according to the invention is connected by known method, advantageously precisely connected, linked, integrated or built to the 4 appropriate part of the dosing device, advantageously to a syringe, especially advantageously to the ISO 594 standard Luer Slip or Luer Lock tip of the syringe, it can be also a part of the dosing device. The advantageous solutions are illustrated in FIGS. 2 and 3.

The essential of the catheter-free, for both genders, adults and children, advantageously for women, men and children applicable process according to the invention is, that after fitting, connecting or building the adapter according to the invention to the appropriate part of the dosing device advantageously to the ISO 594 standard Luer Slip or Luer-lock tip of syringe by known method, the active and/or contrast agent is injected leakage-free into the urethra and/or into the bladder through the external urethral orifice and the urethra, using overpressure, advantageously low overpressure, especially advantageously maximum pressure of 60 water cm for men, male patients and maximum pressure of 30 water cm for women, female patients

As an advantageous solution of the invention the active and/or contrast agent is injected, advantageously slowly injected into the urethra and/or bladder through the external urethral orifice painlessly or by reduced pain, avoiding lesions of urethra, further advantageously injected using gravity pressure, especially advantageously using an infusion device, further advantageously pressing out from balloon or tube, using manual pressure by a method as above, so, that the active agent advantageously active agent with curative effect or drug, further advantageously diagnostic agent comprising contrast agent should remain in appropriate agent concentration of active ingredient or diagnostic agent also in the urethra in its full length.

The leakage-free, catheter-free process, injection is implemented by a method, that the adapter according to the invention fitted or connected by known method—defined and advantageously defined as above—to the dosing device or built on the dosing device advantageously to the syringe as above, is squeezed to the external urethral orifice, and during injection the leakage, flow is inhibited by the help of the 2 sealing collar of the 6 adapter.

A primary basic condition of the leakage-free, catheter-free process, injection, is the steady visibility of the adapter as placed in the urethral orifice whereby any leakage or flow can be detected in due time and eliminated by changing the axial direction and/or the force of squeeze. The visibility depends on the “h” size of characteristic length of the 3 cylindrical connection part (FIG. 1, side-view), which size should be advantageously at least 20 mm, especially advantageously 20 mm.

Further condition of the leakage-free, catheter-free process is, that the dosing device, advantageously syringe or infusion device can be used for injection, of active and/or contrast agent advantageously agents defined as above into the bladder through the urethra for local treatment or diagnosis of bladder and/or urethra and also for ensuring of the appropriate over-pressure.

In addition the process according to invention avoids the superficial lesions and bleedings of the urethra or the mucosa covering the surface of urethra and so the process for the patient is non-invasive painless or pain-reduced.

The prevention, reducing and avoiding the pain of urethra caused by the intervention itself, and reducing the fear of treatment at the same time results a better relaxation, which renders the treatment be better tolerable for the patient because reduced overpressure is needed for the injection of agent.

Further advantage of the catheter-free, non-invasive, humane, leakage-free process according to the invention is, that the agents listed above advantageously agents with curative effect or drug, further advantageously agent, which can be injected comprising diagnostic agent, advantageously liquid, advantageously solution evolve their effect in the urethra itself also, increasing the efficiency of the therapeutic process or diagnostic usage.

Based on the above the subject matter of our invention furthermore is a non-invasive, humane, catheter-free process for painless or pain-reduced and leakage-free injection of active and/or contrast agent in liquid, fluid-like, gas or colloid disperse form, advantageously in aerosol, emulsion, gel, foam or suspension form into the urethra or into the urethra and the bladder through the external urethral orifice by using overpressure, advantageously by using manual or gravity pressure in the dosing device, without using catheter.

During the process according to the invention the 3 connecting part of the 6 adapter is connected by known method to the 4 appropriate part of a dosing device, advantageously of a syringe, further advantageously to an infusion device containing the active and/or contrast agent, advantageously to the ISO 594 standard Luer Slip or Luer-Lock tip of a syringe, then the active and/or contrast agent is injected, advantageously slowly injected, leakage-free into the urethra or into the urethra and the bladder through the 6 adapter, then through the external urethral orifice by inserting the 1 tip-part of the adapter into the external urethral orifice and squeezing the 2 sealing collar of the adapter in a proper way to the external urethral orifice.

Using this method the therapeutic and diagnostic agents, defined and advantageously defined as above and as further on for the indications and treatments depending on the injected agents, defined and advantageously defined as above and as further on, can be injected painlessly or by pain-reduced into the urethra and/or into the bladder, without using catheter, any lesion, so that the agent, advantageously liquid or gas, or different agents of colloid disperse systems in appropriate concentration of active agent and/or diagnostic agent, advantageously contrast agent should remain also in the urethra in its full length, to evolve there also their therapeutic and diagnostic effect.

This causes a gradual increase of the pressure in the urethra and when reaching a certain pressure (retrograde sphincter opening pressure) the sphincter opens and the agent, advantageously the liquid flows into the bladder. This pressure is about 60 cm water column for men, males, and for women, females the average pressure is 30 cm water column lower. These values can be reduced significantly by injection of, advantageously by slowly injection of the agent, advantageously liquid, advantageously solution with curative effect or drug solution, and so by avoiding pain in the urethra and reducing the fear of the treatment and by deliberate relaxation. Thus steadily low pressure (30 to 40 cm water column) expands the urethra to a significant lover extent causing less discomfort and pain.

The adapter and process according to the invention can be used advantageously for injection of active and/or contrast agents, especially advantageously solution of curative active ingredient or of drug, or of diagnostic agent, advantageously contrast agent into the urethra or into the urethra and the bladder through the external urethral orifice for local treatment of bladder and/or urethra, further advantageously for bladder filling, bladder rinsing, especially advantageously for intravesical (bladder) instillation or further advantageously for diagnosis.

Furthermore the adapter and process according to the invention can be used advantageously for injection active and/or diagnostic agent advantageously contrast agent in liquid, fluid-like, gas or colloid disperse form especially advantageously in aerosol, emulsion, gel, foam or suspension form into the urethra and/or bladder through the external urethral orifice for

local treatment of bladder and/or urethra especially advantageously local treatment of interstitial cystitis, bladder pain syndrome, further especially advantageously with analgesic effect, further especially advantageously for local anesthesia, further especially advantageously for local treatment of low urinary tract symptoms, further especially advantageously for local analgesic after-treatment of patients irradiated for prostate cancer, bladder cancer or pelvis tumor, further especially advantageously for local chemotherapeutical treatment of female patients suffering in bladder cancer.

Because of the short urethra the injection of active agents for local chemotherapeutical treatment of bladder can be used only in case of female patients. Because of the larger size of the urethra of male patients and the danger of lesion of the urethra this application cannot be used by men.

Some advantages of the invention are:

1. Using the adapter according to the invention by injection of the solution with curative effect or drug solution or diagnostic solution substantially reduces the inconveniences and pain brought about by the treatment.

2. The catheter-free injection, completely prevents larger and/or smaller mucosal lesions and bleedings of the urethra and serious iatrogenic lesions, as e.g. tardive urethral stricture or chronic bacterial urethritis can be prevented.

3. In the course of treatment only the sterile agent, advantageously solution gets into the urethra and into the bladder thus the risk of bacterial infection and of developing low urinary symptoms is reduced.

4. The process is simple, requires less equipment than bladder filling, bladder instillation with catheter, therefore the treatment without catheter is faster and is less expensive at the same time.

5. As the drug solution is injected into the bladder through the urethra therefore drug solution with unchanged concentration—here not diluted by the urine—remains also in the entire length of the urethra until the following urination (for 2 to 3 hours). Therefore the treatment is more effective, the patients are getting sooner symptom-free and the diagnosis of any urethral irregularity or lesion gets easier.

6. The treatment or diagnosis of urethra is solely by the catheter-free process according to the invention is possible. By a process using a catheter the treatment of urethra is not possible at all.

7. The cost of production of the adapter and therefore its price is significantly lower than the common price of draining catheter plus of the lubricant gel for the insertion of catheter. Therefore the costs of a process without using catheter and also the gel for the catheter are significantly lower than the present practice using the process with catheter.

Thus, using the adapter and process, catheter-free, painless or pain-reduced and leakage-free bladder filling, bladder rinsing, bladder diagnose, advantageously intravesical (bladder) instillation can be implemented without using catheter. By using the adapter and catheter-free process according to the invention, direct local treatment and diagnose of the urethra can be implemented, which is impossible by using catheter.

1. An adapter for use in injection of an active and/or contrast agent in liquid, fluid-like, gas or colloid disperse form, into urethra or into a urethra and a bladder through an external urethral orifice of the urethra comprising:
a central, longitudinal inner through hole,
a rounded off tip-part configured to be inserted into the external urethral orifice,
a sealing collar, and
a cylindrical connecting part connectable by a known method to the to a dosing device,
wherein said injection can be made using said adapter and without using a catheter, painlessly or with reduced pain as compared to use of the catheter, and without leakage.

2. The adapter according to claim 1 wherein the form of the end of the tip-part to be inserted into the external urethral orifice is cylindrical.

3. The adapter according to claim 1 wherein the form of the end of the tip-part to be inserted into external urethral orifice is conical.

4. The adapter according to claim 1 wherein the dosing device is a syringe and the adapter is a syringe adapter.

5. The adapter according to claim 1 wherein the appropriate part of the dosing device is the ISO 594 standard Luer Slip tip of a syringe.

6. The adapter according to claim 1 wherein the appropriate part of the dosing device is the ISO 594 standard Luer Lock tip of a syringe.

7. The adapter according to claim 1 wherein the cylindrical connecting part of the adapter precisely fits, is connected or built to the appropriate part of the dosing device or is part of the dosing device by a known method.

8. The adapter according to claim 1 wherein the solid, pharmaceutically applicable material of the adapter is for human therapy applicable, rigid or flexible material.

9. The adapter according to claim 8 wherein the solid, pharmaceutically applicable material of the adapter is metal, glass, plastic or a natural based material or any alloy or mixture of them.

10. The adapter according to claim 9 wherein the solid, pharmaceutically applicable material of the adapter is copper, bronze, aluminum or stainless steel or any alloy of them, or thermosetting, thermoplastic or flexible plastic or thermoset elastomer or thermoplastic elastomer or latex or latex based, or synthetic or natural based rubber or rubber based or rubber like material or rubber like material based material or any mixture of them.

11. The adapter according to claim 8 wherein the solid, pharmaceutically applicable material of the adapter is plastic, or any alloy or mixture of the materials.

12. The adapter according to claim 11 wherein the pharmaceutically applicable, solid material of the adapter is thermoplastic polyurethane, polytetrafluoroethylene (Teflon, PTFE), polyethylene, polypropylene, polystyrene, polyester, polylactic acid, polycarbonate, polyvinyl chloride, polyether-sulfone, polyacrylate, hydrogel (acrylate), polysulfone, polyetheretherketone (PEEK), polypxylylene (parylene), fluoropolymer, or silicone or any mixture of them.

13. The adapter according to claim 8 wherein the pharmaceutically applicable, solid material of the adapter is for mass producing, for quantity production in series suitable material which can be used for producing the adapter.

14. The adapter according to claim 13 wherein for its production usable mass producing, quantity production in series is die casting technology, or 3D printing.

15. The adapter according to claim 1 wherein at the end of the tip-part to be inserted into the external urethral, the tip of the inner through hole are softened by fine machining, so that no sharp edges causing mucosal lesion remain and the round off and softening are outward and inward towards to the inner through hole achieved also and so the tip part rounded of and softened fits perfectly and generally to the external urethral orifice.

16. The adapter according to claim 1 wherein the “a” length of the tip-part to be inserted into the external urethral orifice, is 8 mm to 12 mm, and its “b” diameter is 6 mm (18 French) corresponding to an average catheter, and the “c” diameter of the central, longitudinal inner through hole of the tip-part is 1.8 mm to 3.5 mm differing from the “f” inner caliber of inner through hole of cylindrical connecting part.

17. The adapter according to claim 16 wherein the “a” length of tip part to be inserted into the external urethral orifice is 10 mm, and the “c” diameter of the central, longitudinal inner through hole of the tip-part is 2.5 mm.

18. The adapter according to claim 1 wherein the round-off radius at the tip part's end is no single data, its tolerance is 1 mm downwards (smaller) or 2 mm upwards (bigger).

19. The adapter according to claim 1 wherein the sealing collar of the adapter is a disk with “d” diameter of 12 mm to 20 mm, and with “e” thickness of 2 mm to 5 mm.

20. The sealing collar according to claim 19 wherein the edges of the disk are chamfered to 45° or rounded off in a circle with 1 mm radius, and the remained edges of the disk are softened, and the value of “d” diameter of the disk is 15 mm and of the “e” thickness is 3 mm.

21. The sealing collar according to claim 19 wherein the external circumference of the disk is knurled.

22. The adapter according to claim 1 wherein the value of “f” inner caliber of the central, longitudinal inner through hole of the cylindrical connecting part is 3.8 mm to 5.2 mm, differing from the “c” diameter of inner through hole of tip part and the outer “g” diameter of the cylindrical connecting part is at least 7 mm.

23. The adapter according to claim 1 wherein the cylindrical connecting part fits precisely or is connected or built to the appropriate part of the dosing device by known method, or it is part of the dosing device.

24. The adapter according to claim 1 wherein the “h” length of the connecting part is at least 20 mm.

25. The adapter according to claim 22 wherein the value of “f” inner caliber of inner through hole is 4.2 mm, the “h” length of cylindrical connecting part is 20 mm, and the “g” outer diameter is 7 mm.

26. Non-invasive, humane process for painless or pain-reduced, leakage-free injection of active and/or contrast agent in liquid, fluid-like, gas or colloid disperse form, into a patient's urethra or into the urethra and a bladder through an external urethral orifice without using a catheter, comprising:
connecting a cylindrical connecting part of the adapter according to claim 1 to a dosing device containing an active and/or contrast agent,
inserting the tip-part into the external urethral orifice, and
squeezing the sealing collar in a proper way to the external urethral orifice, and
injecting the active and/or contrast agent into the urethra or into the urethra and the bladder through the adapter, then through the external urethral orifice by using overpressure in the dosing device.

27. The process according to the claim 26, wherein the process is applicable for both genders, adults and children.

28-30. (canceled)

31. The process according to claim 26, wherein the injection of active and/or contrast agent into the urethra and/or bladder through the external urethral orifice is implemented without a catheter and without using lubricant gel for insertion of the catheter.

32. (canceled)

33. The process according to claim 26, wherein the injection of active and/or contrast agent into the urethra and/or bladder through the external urethral orifice by using the seal collar is leakage-free.

34. The process according to claim 26, wherein the injection of active and/or contrast agent into the urethra and/bladder or through the external urethral orifice is implemented such that the active and/or contrast agent should remain in appropriate agent concentration of active ingredient or diagnostic agent also in the urethra in its full length.

35. The process according to claim 26, wherein the leakage, flow is inhibited by the help of the sealing collar of the adapter by changing the axial direction of dosing device and/or the force of squeeze.

36. The process according to claim 26, wherein during the process the adapter is steadily visible, because the appropriate “h” length of the connecting part of the cylindrical connection part of the adapter makes possible that the adapter inserted in the urethral orifice and so also the process itself can be visually controlled.

37. The process according to claim 26, wherein the overpressure used in dosing device for injection is maximum 60 water cm for men

38. The process according to claim 26, wherein the overpressure used in dosing device for injection is maximum 30 water cm for women.

39. The process according to claim 37, wherein the overpressure used in dosing device for injection is 30 to 40 water cm.

40. (canceled)

41. The process according to claim 26 wherein the injection is implemented by using gravity or manual overpressure in dosing device.

42. The process according to claim 41 wherein the injection is implemented by infusion device.

43. The process according to claim 26 wherein the injected agent is in liquid, fluid-like, gas or colloid disperse form.

44. The process according to claim 43 wherein the injected agent is in aerosol, emulsion, gel, foam or suspension form.

45. The process according to claim 43 wherein the injected agent is in liquid form.

46-62. (canceled)
